(19) United States
(12) Patent Application Publication
(10) Pub. No.: US 2023/0146273 A1
(43) Pub. Date: May 11, 2023

(54) METHOD AND FACILITY FOR PRODUCING A TARGET COMPOUND

(71) Applicant: LINDE GMBH, Pullach (DE)

(72) Inventors: Andreas MEISWINKEL, Rimsting (DE); Hans-Jörg ZANDER, München (DE); Ernst HAIDEGGER, Riemerling (DE); Isabel KIENDL, München (DE)

(21) Appl. No.: 17/627,804

(22) PCT Filed: Jul. 16, 2020
(86) PCT No.: PCT/EP2020/070203

§ 371 Date: Jan. 17, 2022

(30) Foreign Application Priority Data
Jul. 18, 2019 (DE) 10 2019 119 543.8

(51) Int. Cl. C07C 45/50; C01B 3/48; C01B 3/50; B01J 19/24; C07C 45/78; C07C 29/141; C07C 1/24; C07C 2/82

(52) U.S. Cl. CPC C07C 45/50; B01J 19/245; C01B 3/48; C01B 3/506; C07C 1/24; C07C 2/82; C07C 29/141; C07C 45/78; C01B 2203/06; C01B 2203/046; C01B 2203/0238; C01B 2203/0283; C01B 2203/0475; C01B 2203/1241

(57) ABSTRACT

Disclosed is a method for the producingtiona target compoundby oxidative coupling of methane. A starting gas mixture is provided which contains an olefin, carbon monoxide, carbon dioxide and optionally hydrogen, wherein the olefin is subjected to hydroformylation with the carbon monoxide and the hydrogen of the starting mixture to obtain an aldehyde, wherein the paraffin and the olefin have a carbon chain with a first carbon number and the aldehyde has a carbon chain with a second carbon number which is greater by one than the first carbon number. The carbon dioxide present in the starting mixture is removed upstream and/or downstream of the hydroformylation. The carbon dioxide is subjected to dry reforming with methane to obtain carbon monoxide, and that the carbon monoxide subjected to hydroformylation comprises at least part of the carbon monoxide obtained in the dry reforming .

### RELATED APPLICATIONS

The present application is a national stage application under 35 U.S.C. § 371 of International Application No. PCT/EP2020/070203, filed 16 Jul. 2020, which claims priority to German Patent Application No. 10 2019 119 543.8, filed 18 Jul. 2019. The above referenced applications are hereby incorporated by reference in their entirety.

### BACKGROUND

The present invention relates to a method for producing a target compound, in particular propylene, and to a corresponding installation in accordance with the preambles of the independent claims.

The project that has led to the present patent application was promoted within the framework of the financial aid agreement no. 814557 of the European Union’s Research and Innovations program Horizon 2020.

### PRIOR ART

The production of propylene (propene) is described in the specialist literature, for example in the article “Propylene” in Ullmann’s Encyclopedia of Industrial Chemistry, ed. 2012. Propylene is conventionally produced by steam cracking hydrocarbon feeds and conversion processes in the course of refinery processes. In the latter processes, propylene is not necessarily formed in the desired amount and only as one of several components in a mixture with further compounds. Other processes for producing propylene are also known, but are not satisfactory in all cases, for example in terms of efficiency and yield.

An increasing demand for propylene (“propylene gap”), which requires the provision of corresponding selective methods, is predicted for the future. At the same time, it is necessary to reduce or even prevent carbon dioxide emissions. As a potential feedstock, on the other hand, large amounts of methane are available, which are currently only fed to a material utilization in a very limited manner and are predominantly burned.

The object of the present invention is to provide a method for the production of propylene, which is improved in particular in view of these aspects, but also for the production of other organic target compounds, in particular of oxo compounds, such as aldehydes and alcohols with a corresponding carbon backbone.

### DISCLOSURE OF THE INVENTION

Against this background, the present invention proposes a method for producing a target compound, in particular propylene, and a corresponding installation with the respective features of the independent patent claims. Preferred embodiments of the present invention are the subject matter of the dependent claims and of the following description.

In principle, in addition to the aforementioned steam cracking processes, a plurality of different methods exist for converting hydrocarbons and related compounds into one another, some of which will be mentioned below by way of example.

For example, the conversion of paraffins to olefins of identical chain length by oxidative dehydrogenation (ODH, also referred to as ODHE in the case of ethane) is known. The production of propylene from propane by dehydrogenation (PDH) is also known and represents a commercially available and established process. The same also applies to the production of propylene from ethylene by olefin metathesis. This process requires 2-butene as an additional reagent.

Lastly, so-called Methane to Olefins or Methane to Propylene (MTO, MTP) methods exist in which synthesis gas is first produced from methane and the synthesis gas is then reacted to give olefins, such as ethylene and propylene. Corresponding processes can be operated on the basis of methane, but also on the basis of other hydrocarbons or carbon-containing starting materials, such as coal or biomass.

However, ethylene can also be produced by oxidative coupling of methane (OCM), as is the case in one embodiment of the invention and explained below.

The present invention is suitable in principle for use with all methods in which a product mixture is formed which, in addition to an olefin, for example ethylene, additionally contains carbon dioxide and/or carbon monoxide (which can optionally be converted into one another by a water gas shift) in significant amounts, for example, a content of 1 to 30 mole percent, especially of 1 to 20, of 1 to 15 or of 5 to 10 mole percent. A corresponding gas mixture can also comprise in particular methane and/or a paraffin, in particular a paraffin with the same chain length as the olefin. It is referred to here as the “starting gas mixture” as the basis of the method described herein. In the following, predominantly the oxidative coupling of methane is used as an example for such a method; however, the invention is not limited to this.

In accordance with a particularly preferred embodiment of the present invention, the oxidative coupling of methane to provide the starting mixture is thus used, so that it is to be explained in more detail first. The oxidative coupling of methane is described in the literature, for example in J.D. Idol et al., “Natural Gas” in: J.A. Kent (ed.), “Handbook of Industrial Chemistry and Biotechnology”, Volume 2, 12^{th} Edition, Springer, New York 2012.

In accordance with the current state of knowledge, the oxidative coupling of methane comprises a catalyzed gas phase reaction of methane with oxygen, in which one hydrogen atom is split off from each of two methane molecules. Oxygen and methane are activated on the catalyst surface in the process. The resulting methyl radicals first react to give an ethane molecule. In the reaction, a water molecule is further formed. In the case of suitable ratios of methane to oxygen, suitable reaction temperatures and the choice of suitable catalysis conditions, an oxydehydrogenation of the ethane to ethylene is subsequently effected, ethylene being target compound in the oxidative coupling of methane. In this case, a further water molecule is formed. The oxygen used is typically converted completely in the aforementioned reactions.

The reaction conditions in the oxidative coupling of methane conventionally include a temperature of 500 to 900° C., a pressure of 5 to 10 bar and high space velocities. Recent developments in particular are also moving in the direction of the use of lower temperatures. The reaction can take place homogeneously and heterogeneously in the fixed bed or in the fluidized bed. In the oxidative coupling of methane, it is also possible to form higher hydrocarbons having up to six or eight carbon atoms, but the focus is on ethane or ethylene and possibly also propane or propylene.

In particular, due to the high binding energy between carbon and hydrogen in the methane molecule, the yields in the oxidative coupling of methane are comparatively low. Typically, no more than 10 to 15% of the methane used is converted. In addition, the comparatively harsh reaction conditions and temperatures which are required for the cleavage of these bonds also promote the further oxidation of the methyl radicals and other intermediates to carbon monoxide and carbon dioxide. The use of oxygen in particular plays a dual role here. Thus, the methane conversion is dependent on the oxygen concentration in the mixture. The formation of by-products is coupled to the reaction temperature, since the total oxidation of methane, ethane and ethylene is preferably carried out at high temperatures.

Although the low yields and the formation of carbon monoxide and carbon dioxide can be counteracted partly by the choice of optimized catalysts and adapted reaction conditions, a gas mixture formed in the oxidative coupling of methane comprises predominantly unreacted methane and carbon dioxide, carbon monoxide and water besides the target compounds such as ethylene and optionally propylene. Any possible non-catalytic cleavage reactions may also contain considerable amounts of hydrogen. In the terminology used here, such a gas mixture is also referred to as “product mixture” of the oxidative coupling of methane, although it predominantly does not comprise the desired products, but also the unreacted reactant methane and the by-products just explained.

In the oxidative coupling of methane, reactors can be used in which a non-catalytic zone is connected downstream of a catalytic zone. The gas mixture flowing out of the catalytic Zone is transferred into the non-catalytic Zone, where it is initially still present at the comparatively high temperatures which are used in the catalytic Zone. In particular, due to the presence of the water formed in the oxidative coupling of methane, the reaction conditions are similar here to those of conventional steam cracking processes. Therefore, ethane and higher paraffins can be converted to olefins here. Further paraffins can also be fed into the non-catalytic Zone, so that the residual heat of the oxidative coupling of methane can be utilized in a particularly advantageous manner.

Such a targeted steam cracking in a non-catalytic Zone downstream of the catalytic Zone is also referred to as “Post Bed Cracking”. For this, the term “post-catalytic steam cracking” is also used below. If it is stated below that a starting gas mixture used according to the invention is formed or provided “using” or “with the use of” an oxidative coupling of methane, this statement is not to be understood in such a way that only the oxidative coupling itself must be used exclusively in the provision. Rather, the provision of the starting gas mixture can also comprise further process steps, in particular a post-catalytic steam cracking.

In accordance with particularly preferred embodiments of the present invention, paraffins, in particular ethane, which can be separated from any material streams at a suitable point or can be contained in corresponding material streams, can be recycled alone or together with further components for post-catalytic steam cracking. The separation, if carried out, is carried out at a technically suitable separation position, i.e. at a position, at which the separation can be carried out in a particularly uncomplicated and in particular non-cryogenic manner. If it is stated below that ethane or another paraffin other than methane is “recycled into the method”, this may mean in particular a recycling into post-catalytic steam cracking. Methane which is “recycled into the method”, on the other hand, is supplied in particular to the oxidative coupling of methane as a feed. However, recirculation can also take place together and in particular together with carbon monoxide into the oxidative coupling overall.

Steam reforming and dry reforming as well as modifications thereof, including a downstream water gas shift for adjusting the ratio of hydrogen to carbon monoxide, are likewise known as individual technologies.

Hydroformylation is another technology which is used in particular for the production of oxo compounds of the type mentioned at the outset. Propylene is typically reacted in the hydroformylation, but higher hydrocarbons, in particular hydrocarbons having six to eleven carbon atoms, can also be used. The reaction of hydrocarbons having four and five carbon atoms is also possible in principle, but is of lower practical impact. Hydrogenation can follow the hydroformylation in which aldehydes can initially be formed. Alcohols formed by such hydrogenation can be subsequently dehydrated to give the respective olefins.

In Green et al., Catal. Lett. 1992, 13, 341, a process for the production of propanal from methane and air is described. In the process presented, low yields based on methane are generally observed. In the process, oxidative coupling of methane (OCM) and partial oxidation of methane (POX) to hydrogen and carbon monoxide are carried out, which are then followed by hydroformylation. The target product is the aforementioned propanal which has to be isolated as such. A limitation arises from the oxidative coupling of methane to give ethylene, for which, at present, typically only lower conversions and limited selectivities are achieved. Differences of the method described in Green et al. in relation to the present invention are explained below with reference to the advantages that can be achieved according to the invention.

The hydroformylation reaction in the aforementioned method is carried out on a typical catalyst at 115° C. and 1 bar in an organic solvent. The selectivity with respect to the (undesirable) by-product ethane is in the range of approx. 1% to 4%, whereas the selectivity with respect to propanal should achieve more than 95%, typically more than 98%. Extensive integration of process steps or the use of the carbon dioxide formed in large amounts as a by-product, in particular in the oxidative coupling of methane, is not described further here, and so there are thus disadvantages compared with conventional processes. Since partial oxidation is used in the process as a downstream step for oxidative coupling, that is to say there is a sequential interconnection, large amounts of unreacted methane in the partial oxidation have to be managed or separated off in a complex manner from the oxidative coupling.

US 6,049,011 A describes a process for the hydroformylation of ethylene. The ethylene can in particular be formed from ethane. Besides propanal, propionic acid can also be produced as the target product. Dehydration is also possible. However, this document does not disclose any further integration and does not disclose any meaningful utilization of the carbon dioxide formed.

### Advantages of the Invention

Against this background, the present invention proposes a method for preparing a target compound, in particular propylene, in which a starting gas mixture comprising an olefin, in particular ethylene, carbon monoxide and carbon dioxide, is provided. The provision of the starting gas mixture may in particular comprise subjecting a paraffin, in particular methane, to a method in which said components in the starting gas mixture are formed from precursor or starting compounds. In a particularly preferred embodiment, the method can comprise that methane with oxygen is subjected to an oxidative coupling to obtain an olefin, in particular ethylene and, as minor compounds, the further components mentioned. The starting gas mixture can in particular also comprise methane and a paraffin with the same chain length as the olefin, especially ethane. The starting mixture typically also comprises water. Hydrogen can also be present in the starting mixture. However, the presence of hydrogen is not a requirement, even if a corresponding starting mixture should be described in the following as containing hydrogen. The oxidative coupling can also be carried out, for example, without the presence or formation of hydrogen.

As already mentioned at the outset, the oxidative coupling of methane is a method which is, in principle, known from the prior art. In the context of the present invention, known process concepts can be used for the oxidative coupling of methane.

In embodiments of the present invention, (substantially) pure methane or natural gas and/or associated gas fractions of different purification stages up to corresponding raw gas can be used as methane feedstock for oxidative coupling. For example, natural gas can also be fractionated, wherein, when an oxidative coupling is used, methane can be fed into the oxidative coupling itself and higher hydrocarbons can preferably be fed into post-catalytic steam cracking. Oxygen is particularly preferred as oxidizing agent in a corresponding method. Air or oxygen-enriched air can in principle be used also, but lead to nitrogen entry into the system. A separation at a suitable location in the process would in turn be comparatively complicated and would have to be carried out in a cryogenic manner.

In the oxidative coupling, in the embodiments of the present invention, in which it is used, a diluent medium, preferably steam, but also for example carbon dioxide, may be used, in particular to moderate the reaction temperatures. Carbon dioxide can also serve (partially) as oxidizing agent. Compounds that are in principle also suitable as diluents, such as nitrogen, argon and helium, again require complex separation. However, in the current state of the technology, especially recycled methane serves as diluent, which is converted only to a relatively small proportion.

In embodiments of the present invention, the oxidative coupling can be carried out in particular at an overpressure of 0 to 30 bar, preferably 0.5 to 5 bar, and a temperature of 500 to 1100° C., preferably 550 to 950° C. In principle, catalysts known from technical literature can be used, see, for example, Keller and Bhasin, J. Catal. 1982, 73, 9, Hinsen and Baerns, Chem. Ztg. 1983, 107, 223, Kondrenko et al., Catal. Sci. Technol. 2017, 7, 366-381. Farrell et al., ACS Catalysis 6, 2016, 7, 4340, Labinger, Catal. Lett. 1, 1988, 371, as well as Wang et al., Catalysis Today 2017, 285, 147.

In the context of the present invention, the conversion of methane in the oxidative coupling can be in particular more than 10%, preferably more than 20%, particularly preferred more than 30% and in particular up to 60% or 80%. However, the particular advantage of an embodiment of the present invention in which oxidative coupling is employed is not primarily in the increased yield, but in the fact that, additionally, in particular, a relatively high relative proportion of carbon monoxide with respect to ethylene in the product mixture of oxidative coupling, i.e., the starting gas mixture employed in the context of this embodiment, can be utilized.

Typical by-products of the oxidative coupling of methane are carbon monoxide and carbon dioxide formed in the low to two-digit percentage range. A typical product mixture of the oxidative coupling of methane has, for example, the following mixture components:

| | |
|---|---|
| Hydrogen | 0.1 to 10 mole percent |
| Methane | 20 to 90 mole percent |
| Ethane | 0.5 to 30 mole percent |
| Ethylene | 5 to 60 mole percent |
| Carbon monoxide | 0.5 to 30 mole percent |
| Carbon dioxide | 0.5 to 30 mole percent |

These data relate to the dry portion of the product mixture, which may also contain water vapor in particular. Further components such as higher hydrocarbons and aromatics can be present in concentrations of typically less than 5 mole percent, in particular less than 1 mole percent, oxygenates, i.e. aldehydes, ketones, ethers, etc., may be present in traces, i.e. typically less than 0.5 mole percent, in particular less than 0.1 mole percent, in the product mixture of the oxidative coupling.

As already mentioned, in addition to the oxidative coupling of methane, however, other sources are also conceivable in principle for providing the starting gas mixture.

In the context of the present invention, the olefin is subjected to a hydroformylation with carbon monoxide and hydrogen in at least a portion of the starting gas mixture to obtain an aldehyde. Carbon dioxide contained in the starting mixture is separated at least in part upstream and/or downstream of the hydroformylation, i.e., from the starting gas mixture or a portion thereof and/or from a product mixture of the hydroformylation or a portion thereof. This carbon dioxide is at least partially transferred into the product mixture of the hydroformylation if it is not separated upstream.

Processes for hydroformylation are also known in principle from the prior art. In recent times, in corresponding processes, as described in the literature cited below, Rh-based catalysts are typically being used. Older methods also employ Co-based catalysts.

For example, homogeneous, Rh(I)-based catalysts with phosphine and/or phosphite ligands can be used. These may be monodentate or bidentate complexes. For the production of propanal, reaction temperatures of 80 to 150° C. and corresponding catalysts are typically used. All methods known from the prior art can also be used in the context of the present invention.

The hydroformylation typically operates at a hydrogen to carbon monoxide ratio of 1:1. However, this ratio can be, in principle, in the range from 0.5:1 to 10:1. The Rh-based catalysts used may have an Rh content of from 0.01 to 1.00% by weight, wherein the ligands may be present in excess. Further details are described in the article “Propanal” in Ullmann’s Encyclopedia of Industrial Chemistry, ed. 2012. The invention is not limited by the cited process conditions.

In a further process, as described, for example, in the chapter “Hydroformylation” in Moulijn, Makee & van Diepen, Chemical Process Technology, 2012, 235, a pressure of 20 to 50 bar is used with an Rh-based catalyst and a pressure of 70 to 200 bar is used with a Co-based catalyst. Co also appears to be relevant in metallic form for hydroformylation. Other metals are more or less insignificant, especially Ru, Mn and Fe. The temperature range used in said method is between 370 K and 440 K.

In the process disclosed in the chapter “Synthesis involving Carbon Monoxide” in Weissermel & Arpe, Industrial Organic Chemistry 2003, 135, mainly Co- and Rh-phosphine complexes are used. With specific ligands, hydroformylation can be carried out in aqueous medium and recovery of the catalyst is readily possible.

According to Navid et al., Appl. Catal. A 2014, 469, 357, in principle all transition metals capable of forming carbonyls can be used as potential hydroformulation catalysts, wherein an activity as per Rh > Co > Ir, Ru > Os > Pt > Pd > Fe > Ni is observed according to this publication.

By-products in the hydroformylation are formed in particular by the hydrogenation of the olefin to give the corresponding paraffin, i.e., for example, from ethylene to ethane, or the hydrogenation of the aldehyde to give the alcohol, i.e. from propanal to propanol. In accordance with the article “Propanols” in Ullmann’s Encyclopedia of Industrial Chemistry, ed. 2012, propanal formed by hydroformylation can be used as the main source of 1-propanol in industry. In a second step, propanal can be hydrogenated to give 1-propanol.

In the context of the present invention, the olefin in the starting gas mixture has a carbon chain having a first carbon number, and the aldehyde has a carbon chain having a second carbon number which is one greater than the first carbon number due to the chain extension in the hydroformylation. The present invention is described below predominantly with reference to ethylene as olefin, but can in principle also be used with higher hydrocarbons.

In general, irrespective of the specific type of provision of the starting gas mixture within the scope of the present invention, at least some of the carbon dioxide separated in the manner explained is subjected to a dry reforming with methane to obtain carbon monoxide. Since the content of carbon dioxide in a corresponding starting gas mixture is often only in the single-digit percentage range depending on its production, further carbon dioxide from other sources can be additionally supplied to dry reforming at any time. However, the invention always comprises that the carbon dioxide present in the starting gas mixture, which is separated upstream and/or downstream of the hydroformylation, is at least partly fed to the dry reforming.

Dry reforming is also a method known in principle from the prior art. Reference is made by way of example to Halmann, “Carbon Dioxide Reforming. Chemical fixation of carbon dioxide: methods for recycling CO₂ into useful products”, CRC Press 1993, ISBN 978-0-8493-4428-2, instead of to many. Dry reforming is also referred to as carbon dioxide reforming. In dry reforming, carbon dioxide is reacted with hydrocarbons, such as methane. Here, hydrogen and carbon monoxide and also unreacted carbon dioxide and optionally synthesis gas containing hydrocarbons used are formed, as is conventionally produced by steam reforming. In dry reforming, the reagent steam is replaced to some extent by carbon dioxide. In dry reforming, one molecule of carbon dioxide is reacted with one molecule of methane to give two molecules of hydrogen and two molecules of carbon monoxide. A certain challenge in dry reforming is the comparatively simple further reaction of the formed hydrogen with carbon dioxide to give water and carbon monoxide.

Pressures of up to 40 bar and temperatures of up to 950° C. are typically used in dry reforming. Dry reforming is typically carried out using Ni or Co catalysts or bimetallic catalysts having Ni and Co. Further details are described, for example, in the articles “Gas Production: 2. Processes” and “Hydrogen: 2. Production″ in Ullmann’s Encyclopedia of Industrial Chemistry, ed. 2012, and in the chapter “Synthesis Gas” in Weissermel & Arpe, Industrial Organic Chemistry, 2003, 15. Embodiments, in particular with respect to the catalysts mentioned, can also be found, for example, in San-Jose-Alonso et al., Appl. Catal. A, 2009, 371, 54, and Schwab et al., Chem. Ing. Tech. 2015, 87, 347.

As mentioned, in embodiments of the present invention, a hydrogenation and dehydration of the components formed in the hydroformylation can also occur for the production of further products.

Hydrogenation of different unsaturated components is a well known and established technology for converting components having a double bond into the corresponding saturated compounds. Typically, very high or complete conversions with selectivities of well above 90% can be achieved. Typical catalysts for the hydrogenation of carbonyl compounds are based on Ni, as is also described, for example, in the article “Hydrogenation and Dehydrogenation” in Ullmann’s Encyclopedia of Industrial Chemistry, ed. 2012. Noble metal catalysts can also be used specifically for olefinic components. Hydrogenations are part of the standard reactions of technical chemistry, as also shown, for example, in M. Baerns et al., “Beispiel 11.6.1: Hydrierung von Doppelbindungen” [“Example 11.6.1: hydrogenation of double bonds”], Technische Chemie 2006, 439. In addition to unsaturated compounds (understood here are olefins, in particular), the authors also mention other groups of substances, such as, for example, aldehydes and ketones in particular as substrates for hydrogenation. Low-boiling substances such as butyraldehyde from the hydroformylation are hydrogenated in the gas phase. Here, Ni and certain noble metals, such as Pt and Pd, typically in supported form, are used as hydrogenation catalysts.

For example, in the article “Propanols” in Ullmann’s Encyclopedia of Industrial Chemistry, ed. 2012, a heterogeneous gas phase process is described which is carried out at 110 to 150° C. and a pressure of 0.14 to 1.0 MPa at a hydrogen to propanal ratio of 20:1. Reduction takes place with excess hydrogen and the heat of the reaction is dissipated by circulating the gas phases through external heat exchangers or by cooling the reactor in the interior. The efficiency with respect to hydrogen is more than 90%, the conversion of the aldehyde is effected up to 99.9% and alcohol yields of more than 99% result. Widely used commercial catalysts include combinations of Cu, Zn, Ni and Cr supported on aluminum oxide or kieselguhr. Dipropyl ether, ethane and propyl propionate are mentioned as typical by-products which can form in traces. According to the general prior art, the hydrogenation is preferably effected in particular only with stoichiometric amounts of hydrogen or only a low hydrogen excess.

Details of corresponding liquid-phase processes are also given in the literature. These are carried out, for example, at a temperature of 95 to 120° C. and a pressure of 3.5 MPa. Typically, Ni, Cu, Raney nickel or supported Ni catalysts reinforced with Mo, Mn and Na are preferred as catalysts. 1-propanol can be prepared with 99.9% purity, for example. The main problem with the purification of 1-propanol is the removal of water from the product. If, as in one embodiment of the present invention, propanol is dehydrated to give propylene, water is also one of the reaction products in this step, so that water does not have to be removed beforehand. The separation of propylene and water is thus made simple.

Dehydration of alcohols on suitable catalysts to prepare the corresponding olefins is also known. In particular, the production of ethylene (from ethanol) is common and is gaining importance in connection with the increasing production quantities of (bio)ethanol. Commercial use has been achieved by different companies. For example, reference is made to the aforementioned article “Propanols” in Ullmann’s Encyclopedia of Industrial Chemistry and Intratec Solutions′ “Ethylene Production via Ethanol Dehydration”, Chemical Engineering 120, 2013, 29. Accordingly, the dehydration of 1- or 2-propanol to give propene has no practical value so far. Nevertheless, the dehydration of 2-propanol in the presence of mineral acid catalysts at room temperature or above is very easy to carry out. The reaction itself is endothermic and equilibrium limited. High conversions are favored by low pressures and high temperatures. Typically, heterogeneous catalysts based on Al₂O₃ or SiO₂ are used. In general, several types of acid catalysts are suitable and, for example, molecular sieves and zeolites can also be used. Typical temperatures range from 200 to 250° C. for the dehydration of ethanol or 300 to 400° C. for the dehydration of 2-propanol or butanol. Owing to the equilibrium limiting, the product stream is typically separated off (separation of the olefin product and also at least partially of the water, for example by distillation) and the stream containing unconverted alcohol is recycled to the reactor inlet. In this way, overall very high selectivities and yields can be achieved.

Overall, the present invention proposes the coupling of a hydroformylation process and a dry reforming, wherein the hydroformylation and the dry reforming are fed from components of a common starting gas mixture, namely firstly with an olefin, carbon monoxide and optionally hydrogen and secondly with carbon dioxide. In the context of the present invention, particular advantages result in particular from the fact that dry reforming can be carried out with the carbon dioxide as starting material, which is contained in the starting mixture, for example, because it is inevitably formed as a significant by-product in the oxidative coupling of methane, and that the remaining components from the starting mixture and components from a product mixture of the dry reforming, the latter optionally after carrying out a water gas shift, can be used in the hydroformylation without complicated cryogenic separation steps. In particular, paraffins from the starting gas mixture can be carried along in the hydroformylation and subsequent thereto can be separated more easily, or hydrogen formed in the dry reforming can be used for later hydrogenation steps. The unreacted paraffins can in this way be easily recycled and reused in reaction feed, as already explained above with reference to oxidative coupling and post-catalytic steam cracking.

The present invention therefore proposes that the carbon dioxide that originates from the starting gas mixture and that has been separated upstream and/or downstream of the hydroformylation, and was, for example, previously formed in the oxidative coupling as a by-product, is subjected to a dry reforming with methane at least in part to obtain carbon monoxide. In this way, the present invention results in a particularly advantageous and value-adding use of the carbon dioxide contained in the starting gas mixture and which cannot be avoided as a by-product in the preparation of the starting gas mixture. The advantages of the invention thus consist in an advantageous use of a (by-)product of one method in the other and an advantageous use of the products of both methods in a downstream step. As mentioned, the wording according to which “at least the separated carbon dioxide is subjected at least in part with methane to dry reforming to obtain carbon monoxide”, does not preclude that further carbon dioxide provided from any desired source can be supplied to dry reforming. This is the case in one embodiment of the present invention.

By using the present invention, a significant improvement in the carbon dioxide footprint can be achieved overall by using the carbon dioxide in the process (feeding it into the dry reforming). The invention optionally enables an increase in the possible yield of value products in the oxidative coupling by the use of the carbon monoxide as a reaction partner in the hydroformylation. At the same time, in the context of the present invention, the effort required in product purification and separation, in particular by the avoidance of cryogenic separation steps, is reduced. The separation of C2- and C3- components can take place at comparatively moderate temperatures and optionally while avoiding drying. Overall, the energy efficiency is improved and large circuits which are conventionally required due to the limited conversions in the oxidative coupling are avoided or minimized. Non-value added steps such as methanation are avoided in the context of the present invention, as is the formation of by-products and coupling products as in other processes for the production of propylene.

The above-mentioned article of Green et al. already describes the synthesis of propanal from methane and air, wherein a total low yield in relation to methane is reported. In this method, a linking of oxidative methane coupling and partial oxidation is used, followed by hydroformylation. The target product is propanal which must be isolated as such. Here, limitations are the oxidative coupling of methane to ethylene, for which only small conversions and limited selectivities are achieved nowadays. A further Integration of process steps or the use of carbon dioxide produced is not described in Green et al. The advantages that can be achieved according to the invention are thus not given here. A scheme cited in Green et al. lists partial oxidation as a downstream unit for oxidative coupling. Due to this sequential interconnection, large amounts of methane which are not converted in the oxidative coupling are therefore to be dealt with in the partial oxidation. The present invention overcomes this disadvantage by the parallel interconnection of an oxidative coupling or, more generally, the provision of the starting gas mixture with the dry reforming, wherein the resulting material streams are then combined in the required ratio.

At no point in Green et al. is there any mention of dry reforming to produce hydrogen and carbon monoxide; only a recycling of carbon dioxide in an overall recycle for partial oxidation is hinted at. It is proposed here to separate ethylene, carbon dioxide and water from a product stream in a cryogenic manner, so that a residue containing methane, carbon monoxide and hydrogen remains. This is not feasible in practice, since cryogenic separation of carbon dioxide and/or water will result in very rapid displacement by solid carbon dioxide or ice.

As mentioned, further by-products can be formed in a method for providing the starting gas mixture, for example in the oxidative coupling. If suitable, these can be separated, for example together with reaction water, if necessary by condensation and/or water washing from a corresponding product mixture of the oxidative coupling. Owing to its strong interaction with suitable solvents or washing liquids, carbon dioxide can likewise be removed comparatively easily from the product mixture, wherein it is possible to use known methods for removing carbon dioxide, in particular corresponding scrubbing (for example amine scrubbing). The same also applies to a separation downstream of the hydroformylation. Cryogenic separation is not required, so that the entire method of the present invention, at least including dry reforming and hydroformylation, forgoes cryogenic separation steps. Should subsequent steps require the absence of, or only a very low residual concentration of, carbon dioxide (for example due to catalytic inhibition or poisoning), the residual carbon dioxide content after amine scrubbing can be further reduced by an optional caustic scrubbing as fine cleaning, as required.

Any water-containing gas mixtures occurring in the context of the present invention can be subjected to drying at a suitable point in each case. For example, drying can take place downstream of the hydroformylation if, in one embodiment of the present invention, this takes place in the aqueous phase and the hydrogenation downstream of the hydroformylation requires a dry stream as reaction feed. If this is not necessary for the subsequent process steps, drying does not have to take place until complete dryness; rather, water contents can optionally also remain in corresponding gas mixtures, as long as these are tolerable. Different drying steps can also be provided at different points in the method and optionally with different degrees of drying.

The separation of the aforementioned by-products advantageously takes place completely non-cryogenically and is therefore extremely simple in terms of apparatus and in terms of energy expenditure. This represents a substantial advantage of the present invention over prior art methods which typically require complex separation of components that are undesirable in subsequent process steps.

“Non-cryogenic” separation is to be understood as a separation or separation step which is carried out in particular at a temperature level above 0° C., in particular at typical cooling water temperatures of from 5 to 40° C., in particular from 5 to 25° C., optionally also above ambient temperature. In particular, however, non-cryogenic separation in the sense referred to here represents a separation without the use of a C2 and/or C3 cooling circuit and it is therefore carried out above -30° C., in particular above -20° C.

Typically, unreacted methane, a paraffin, in particular ethane, and carbon monoxide are present as components in a corresponding starting gas mixture, in particular if it originates from an oxidative coupling. These compounds can be transferred into the subsequent hydroformylation without difficulty. This is what the present invention provides. Carbon monoxide can be reacted with the olefin together with carbon monoxide from dry reforming. The methane and the paraffin are not converted in the hydroformylation. Since heavier compounds with a higher boiling point or other polarity are formed in the hydroformylation, they can be separated off comparatively easily, and likewise non-cryogenically, from the remaining lighter boiling components. Instead of a complete separation, it is also possible at any time to achieve an enrichment of certain material streams of corresponding components and/or a depletion of other components. All technologies known to the person skilled in the art can be used here, for example absorptive or adsorptive methods, membrane methods and enrichment or separation steps based on organometallic Frameworks.

Where reference is made herein to liquids or gases or corresponding mixtures being rich or poor in one or more components, “rich” shall mean a content of at least 90%, 95%, 99%, 99.5%, 99.9%, 99.99% or 99.999% and “poor” shall mean a content of not more than 10%, 5%, 1%, 0.1%, 0.01% or 0.001% on a molar, weight or volume basis. The term “predominant” refers to a content of at least 50%, 60%, 70%, 80% or 90% or corresponds to the term “rich”. In the terminology used here, liquids and gases or corresponding mixtures can also be enriched or depleted regarding one or more components, wherein these terms refer to a corresponding content in a starting mixture. The liquid or the gas or the mixture is “enriched” when at least 1.1 times, 1.5 times, 2 times, 5 times, 10 times, 100 times or 1000 times the content is present, “depleted”, if at most 0.9 times, 0.5 times, 0.1 times, 0.01 times or 0.001 times the content of a corresponding component is present, based on the starting mixture. A (theoretically possible) complete separation in this sense represents a depletion to zero with respect to a component in a fraction of a starting mixture, which therefore passes completely into the other fraction and is present there enriched. This is also to be included by the terms “enriching” and “depleting”.

If reference is made here that a separation takes place, it is also possible at any time that certain material streams are merely enriched in the corresponding components or depleted in other components. All technologies known to the person skilled in the art can be used here, for example absorptive or adsorptive methods, membrane methods and enrichment or separation steps based on organometallic Frameworks.

In particular, methane and ethane can, as mentioned, be recycled into the method, in particular into the oxidative coupling used in one embodiment of the present invention at the stated points. Ethane does not necessarily need to be recycled to a separate reactor section for post-catalytic steam cracking, but can also be recycled unseparated from the methane in the oxidative coupling process as a whole. The same also applies to remaining carbon monoxide, which can optionally be further oxidized to carbon dioxide in the oxidative coupling.

In a particularly preferred embodiment of the present invention, it is therefore provided that, from at least a portion of a product mixture of the hydroformylation or of a method step following the hydroformylation, i.e. in particular the hydrogenation or dehydration as explained below, a partial mixture is obtained by separating at least a portion of heavier components, which is enriched with respect to the product mixture of the hydroformylation or the method step subsequent to the hydroformylation, at least in methane and a paraffin, in particular ethane, and optionally in carbon monoxide, or is poor in or free of heavier components, wherein “poor” is to be understood here in particular as a content of less than 10, 5, 1, 0.5 or 0.1 mole percent. In one embodiment of this invention, this partial mixture is unseparated and recycled at least to some extent into the method, wherein the recycling takes place in particular into a method step which serves to provide the starting gas mixture. In one embodiment of the present invention, the recycling is effected in a reactor used for performing an oxidative coupling.

In a particularly advantageous development, the present invention can comprise an energy integration, that is to say a coupling of heat flows for endothermic and exothermic reactions. Exothermic reactions are in particular oxidative coupling, hydroformylation and hydrogenation. A reforming aimed at for providing additional hydrogen and the dehydration are, on the other hand, endothermic reactions. In one embodiment of the present invention, in which an oxidative coupling is made, the use of the waste heat from this for dry reforming is advantageous, which takes place in a similarly high temperature range of typically more than 800° C.

In one exemplary embodiment, dry reforming can take place at approximately 15 to 25 bar, depending on the available methane pressure and the preferred operating range. Downstream recompression can therefore be provided, but this can also take place in a common final compressor stage with the product gas of the oxidative coupling. In this way, an oxidative coupling and dry reforming product stream can be passed together through the amine scrubbing to remove carbon dioxide. Although a larger volume flow is to be processed there in this embodiment, it is also possible in this way to remove residual amounts of carbon dioxide in the product gas of the dry reforming. However, residual amounts of carbon dioxide that would interfere with cryogenic separation may be tolerated, if necessary, in embodiments of the present invention where such cryogenic separation does not occur. Therefore, an optionally provided fine purification, for example using a caustic scrubbing, may be dispensed with.

In the context of the present invention, the aldehyde formed in the hydroformylation can be the target compound or, in the context of the present invention, this aldehyde can be further converted to give an actually desired target compound. The latter variant in particular represents a particularly preferred embodiment of the present invention.

In particular, when the aldehyde is reacted to give the target compound, the aldehyde can first be hydrogenated to give an alcohol which has a carbon chain having the second carbon number, i.e. the same carbon number as the aldehyde. A corresponding process variant is particularly advantageous because it is possible to use for this hydrogen which is contained in a product mixture of the dry reforming and which can be already present in a feed mixture upstream of the hydroformylation and can be passed through the hydroformylation.

In the context of the present invention, a content of hydrogen and carbon monoxide in a product mixture of the dry reforming can be adjusted, in particular, in a water gas shift of a type known in principle. The water gas shift can be carried out in particular downstream of the dry reforming and in particular upstream of the hydroformylation. In particular, the water gas shift takes place before a product stream from the dry reforming, and the starting gas mixture or a residue thereof remaining after the separation of carbon dioxide, which possibly may have been carried out, is combined. By using the water gas shift, the present invention enables a precise adaptation of the respective hydrogen and/or carbon monoxide contents to the respective need in the hydroformylation or the downstream hydrogenation.

An appropriate water gas shift can also be used to take account, in particular, of any carbon monoxide content in the starting gas mixture, which is combined with carbon monoxide from the dry reforming to form a feed into the hydroformylation. The use of a water gas shift downstream of the dry reforming enables an exact adaptation to the respective requirements in the hydroformylation.

Hydrogen can be fed in at any suitable point in the method according to the invention and its embodiments, in particular upstream of the optionally provided hydrogenation. In this way, hydrogen is available for this hydrogenation. The feeding need not take place directly upstream of the hydrogenation; rather, hydrogen can also be fed in by method or separation steps present or carried out upstream of the hydrogenation. Hydrogen can also be separated off, for example, from a partial stream of a product stream from the dry reforming or formed as a corresponding partial stream, for example by separation steps known per se, such as pressure swing adsorption.

In accordance with a further embodiment of the present invention, during the reaction of the aldehyde to give the actual target compound of the method according to the invention, the alcohol formed by the hydrogenation is dehydrated to give a further olefin (based on the earlier olefin contained in the starting gas mixture), wherein the further olefin, in particular propylene, has a carbon chain with the second carbon number mentioned, i.e. the carbon number of the aldehyde formed beforehand and of the alcohol formed therefrom.

In particular, the alcohol formed in the reaction of the aldehyde can be separated off comparatively easily from unreacted paraffin. In this way, a recycle stream of the paraffin can also be formed non-cryogenically here and recycled, for example, into the oxidative coupling of methane.

As already mentioned several times, in the context of the present invention, the first carbon number can be two and the second carbon number can be three; therefore, it is first possible, for example, to produce ethylene as olefin from methane in an oxidative coupling, wherein the ethylene is converted to propanal in the hydroformylation. This propanal can subsequently be reacted by a hydrogenation to give propanol and this in turn can be reacted to give propylene by a dehydration.

In a particularly preferred embodiment, the present invention permits the use of all components of natural gas. For this purpose, any natural gas fractions or raw natural gas can be used, as already explained above for the oxidative coupling of methane.

As already mentioned, the carbon monoxide obtained in the dry reforming can be obtained in a product mixture which also contains at least hydrogen. This hydrogen can be passed through the hydroformylation and subsequently used in a hydrogenation. The product mixture from the dry reforming can, as has likewise already been mentioned, be subjected to a water gas shift. In particular, the product mixture from the dry reforming and/or the product mixture from the water gas shift can be subjected, at least partially unseparated, to the hydroformylation.

Further aspects of the present invention have also already been mentioned in principle. In particular, the carbon dioxide can at least partly be separated from the starting gas mixture in a non-cryogenic manner and subjected to dry reforming. The carbon monoxide and the olefin in the remaining residue of the starting gas mixture and optionally further components therein can be subjected to the hydroformylation at least in part without a prior separation from one another. As mentioned, in the context of the present invention, in principle a complete non-cryogenic separation of obtained gas mixtures can be achieved. This is not necessarily the case for the separation of natural gas into the methane fraction and the fraction with heavier hydrocarbons mentioned previously.

As already mentioned, the starting gas mixture can in particular contain methane and at least one paraffin, wherein at least a portion of the methane and the paraffin is able to pass through the hydroformylation without conversion. As mentioned in detail above, this part can be separated off and recycled downstream of the hydroformylation. Depending on expediency, the separation can take place directly downstream of the hydroformylation, i.e. before each process step following the hydroformylation, or downstream of a process step following the hydroformylation, for example after a hydrogenation or dehydration, but also after any separation or treatment steps.

In a particularly preferred embodiment of the present invention, the starting mixture, depending on its provision, in particular after a condensate removal, is compressed to a pressure level at which both the carbon dioxide is separated from the oxidative dehydrogenation before and/or after the hydroformylation and the hydroformylation is carried out. Additional intermediate steps may optionally also be provided between the separation of carbon dioxide and the hydroformylation upstream and/or downstream thereof. Both methods take place essentially at the same pressure level, which means in particular that no additional compression takes place between the two and the precise operating pressure of both steps only results from the process-related pressure losses between the two steps.

The pressure level at which the removal of carbon dioxide and the hydroformylation are operated preferably represents the highest pressure level in the overall process, which means in particular that the dry reforming is carried out at a lower pressure level than the separation of carbon dioxide and the hydroformylation upstream and/or downstream thereof. In this way, it is possible to dispense with providing otherwise required additional compression steps and corresponding compressors.

In the context of the present invention, the starting gas mixture is advantageously provided at the pressure level previously recited for the oxidative coupling of methane, the dry reforming is advantageously carried out at a pressure level of from 10 to 80 bar, in particular from 15 to 50 bar, and the hydroformylation and the removal of carbon dioxide are advantageously carried out at a pressure level of from 15 to 100 bar, in particular from 20 to 50 bar.

The present invention also extends to an installation for producing a target compound, in relation to which reference is expressly made to the corresponding independent patent claim. A corresponding installation, which is preferably set up for carrying out a method, as has been explained above in different embodiments, benefits in the same way from the advantages already mentioned above.

The invention will be explained in more detail below first with reference to the accompanying drawing, which illustrates a preferred embodiment of the present invention. An exemplary embodiment of the invention is subsequently explained in more detail, which is carried out in particular using the method illustrated in the drawing.

### BRIEF DESCRIPTION OF THE DRAWINGS

FIG. 1 illustrates a method according to one embodiment of the invention in the form of a schematic flowchart.

If reference is made below to method steps, such as oxidative coupling of methane, dry reforming or hydroformylation, these are also to be understood to cover the apparatus used in each case for these method steps (in particular, for example, reactors, columns, scrubbing devices, etc.), even if this is not expressly referred to. In general, the explanations relating to the method apply to a corresponding installation in the same way in each case.

### DETAILED DESCRIPTION OF THE DRAWINGS

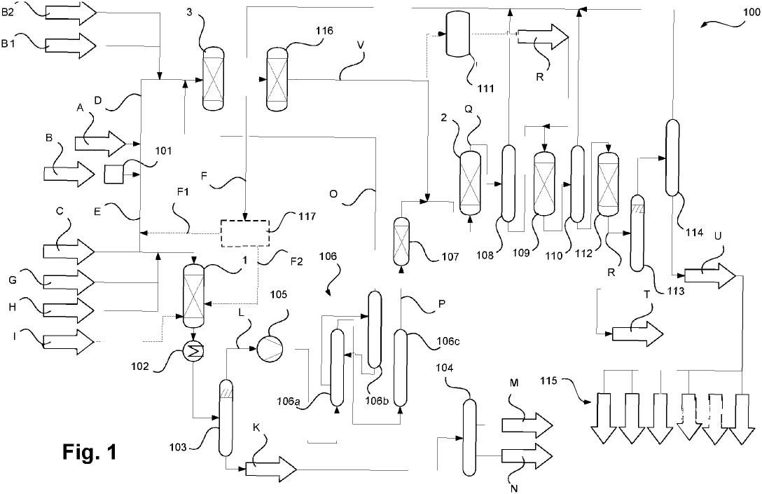

FIG. 1 illustrates a method according to a particularly preferred embodiment of the present invention in the form of a schematic flowchart and is designated overall by 100.

Central method steps or components of the method 100 are an oxidative coupling of methane, which is designated here overall by 1, and a hydroformylation, which is designated here overall by 2. The method 100 further comprises a dry reforming, designated here overall by 3.

In the example shown, a methane stream A is supplied to the method 100. Instead of the methane stream A or in addition to this, a raw natural gas stream B can also be provided. If necessary, the raw natural gas stream B can be prepared by means of any treatment step 101. Partial streams of the methane stream A or of the raw natural gas stream are denoted by D and E. Further, in the example illustrated here, a vapor stream B1 and a carbon dioxide stream B2 are provided from an external source.

The partial stream E is fed together with a partial stream F1 of a recycle stream F (or, as explained below, optionally also together with the entire recycle stream F) into the oxidative coupling 1. In this case, mixing with oxygen, which is provided in the form of a material stream C, and optionally with steam, which is provided in the form of a material stream G, is carried out. The steam of the material stream G, as well as nitrogen of an optionally provided nitrogen stream H, serves as a diluent or moderator and in this way prevents in particular a thermal runaway in the oxidative coupling 1. Water can also make a contribution in order to ensure the catalyst stability (long-term performance) and/or to enable a moderation of the catalyst selectivity.

A reactor used in the oxidative coupling 1 can have a region for performing a post-catalytic steam cracking, as was explained at the outset. A partial stream F2 of the recycle stream F comprising ethane can optionally be fed into this region. Alternatively or additionally, it is also possible to feed a separately provided ethane stream I. A feed of propane can also be provided in principle. The ethane stream I and optionally propane and heavier components can also be separated from raw natural gas, the remainder of which is then provided as methane stream A.

Downstream of the oxidative coupling 1, an aftercooler 102 is provided downstream of which there is, in turn, a condensate separation 103. A condensate stream K formed in the condensate separation 103, which contains predominantly or exclusively water and possibly further, heavier compounds, can be fed to a device 104, in which in particular a (purified) water stream M and a residual stream N can be formed.

The product mixture of the oxidative coupling 1 freed of condensate, here generally referred to as “starting gas mixture”, is compressed in the form of a material stream L in a compressor 105 and subsequently supplied to a carbon dioxide removal, designated overall by 106, which can be carried out, for example, using corresponding scrubbing. In the embodiment shown here, a scrubbing column 106a for an amine scrubbing and the regeneration column 106b for the amine-containing scrubbing liquid loaded with carbon dioxide in the scrubbing column 106a are shown. An optional scrubbing column 106c for fine purification, for example for a caustic scrubbing, is also shown. As mentioned, the removal of carbon dioxide is generally known by corresponding scrubbing and recovery. It is therefore not explained separately. As mentioned several times, a corresponding carbon dioxide removal can also take place downstream.

A carbon dioxide stream O formed in the carbon dioxide removal 106 can, as explained further below, be fed into the dry reforming 3.

A mixture of components remaining in the form of a material stream P, after the removal of carbon dioxide in the carbon dioxide removal 106, contains predominantly ethylene, ethane and carbon monoxide. It is optionally dried in a dryer 107 and then fed to the hydroformylation 2.

In the hydroformylation 2, propanal is formed from the olefins and carbon monoxide, which together with the further components explained is carried out in the form of a material stream Q from the hydroformylation 2. Optionally, unreacted ethane and other lighter boiling compounds such as methane and carbon monoxide can be separated from the material stream Q in a separation 108, which can then be transferred to the recycle stream F. Alternatives to the separation 108 are discussed further below, however, the separation 108 is a preferred embodiment.

In one of a hydrogenation 109, the propanal can be converted to propanol. The alcohol stream is fed to a further separation 110 optionally provided as an alternative to the separation 108, where even more lighter boiling components can be separated off and transferred into the recycle stream F.

The hydrogenation 109 can be operated with hydrogen which is contained in a product stream of the dry reforming 3 and is carried along in the hydroformylation. Alternatively, the separate feeding of required hydrogen in the form of a material stream R is also possible, in particular from a separation of hydrogen in a pressure swing adsorption 111.

A product stream from the hydrogenation 109 or from the optionally provided separation 110 is fed to a dehydration 112. In said dehydration, propylene is formed from the propanol. A product stream R from the dehydration 112 is fed to a condensate separation 113 where it is freed of condensible compounds, in particular water. The water can be carried out of the process in the form of a water stream T. The water streams N and T can, optionally after a suitable work-up, also be fed again to the process for steam generation. In this way, for example, at least a part of the steam flow B1 can be provided.

The gaseous residue remaining after the condensate separation 113 is fed to a further separation 114 optionally provided as an alternative to the separations 108 and 110 where, in particular likewise unreacted ethane and lighter boiling compounds can be separated off and transferred into the recycle stream F. A product stream U formed in the separation 114 can be carried out of the process and used further process steps, for example for the production of plastics or other further compounds, as indicated here overall by 115. Corresponding methods are known per se in a variety of forms and comprise the use of the propylene from the method 100 as intermediate product or starting product in the petrochemical value chain.

Unreacted ethane and other light compounds such as methane and carbon dioxide are recycled into oxidative coupling 1 in the form of a material stream F, as mentioned several times. Optionally, a separation 117 can be provided in which the partial streams F1 and F2 can be formed. In particular, methane and ethane can be separated from one another in this way, wherein the methane in the partial stream F1 in the oxidative coupling 1 can be led to the reactor inlet and the ethane in the partial stream F2 can be led to a reactor zone used for the post-catalytic steam cracking.

A water gas shift 116 is optionally connected downstream of the dry reforming 3. A product mixture V formed in each case in the dry reforming 3 or the (optional) water gas shift 116, which predominantly or exclusively contains hydrogen and carbon monoxide, is fed (after an optional hydrogen separation in the pressure swing adsorption 111), together with the material stream P freed of carbon dioxide, from the oxidative coupling 1 to the hydroformylation 2.

### Exemplary Embodiment

In the context of the present invention, a starting gas mixture was considered, as can basically be provided by means of the oxidative coupling of methane. In particular, the latter has the previously recited component fractions.

As an exemplary composition and as a basis for the following calculation example, the following composition is recited:

| Component | Mole % |
|---|---|
| H₂ | 6.00 |
| CO | 1.50 |
| CO₂ | 3.00 |
| Methane | 72.00 |
| Ethylene | 3.50 |
| Ethane | 1.20 |
| Higher hydrocarbons | 1.50 |
| H₂O | 11.30 |
| Total | 100 |

For an ideal overall reaction of the integrated process proposed in accordance with one embodiment of the present invention downstream of the provision of the starting gas mixture (hydroformylation, hydrogenation and dehydration), the following gross equation is obtained:

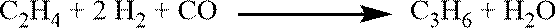

Thus, in the starting gas mixture recited above, the two required equivalents of hydrogen are almost available in this exemplary embodiment and there is only a minor additional requirement. However, only about ⅓ of the stoichiometric demand of carbon monoxide is provided, while at the same time there is a significant amount of carbon dioxide. If it is now possible to convert this amount of carbon dioxide as required into carbon monoxide and if necessary hydrogen, the stoichiometry of the gross reaction equation can be easily met.

In accordance with the invention, this can be achieved by the step of dry reforming, in which the following idealized reaction takes place:

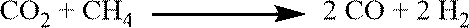

Further reactions also lead to the formation of hydrogen:

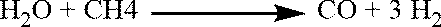

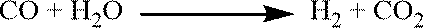

A fine adjustment of the ratio of hydrogen to carbon monoxide is possible if necessary in the optional downstream shift reaction (in equation V from left to right) or reversed shift reaction (in equation V from right to left):

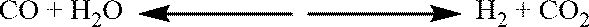

Other embodiments of the oxidative coupling can also lead in particular to a lower hydrogen content in the product gas. Accordingly, the additional provision of hydrogen by the above-mentioned reactions II to V is required. A corresponding provision can take place, for example, by means of classical reforming.

In the following, a calculation example based on the oxidative coupling is recited to document the advantages that can be achieved in accordance with the present invention, in which especially the component proportions required or advantageous for a starting gas mixture are determined.

The gross equation I recited above applies to an ideal overall reaction of the integrated process after oxidative coupling (hydroformylation, hydrogenation and dehydration).

The following explanations build on the exemplary embodiment described above and assume idealized conditions and reactions I to V in a reforming, dry reforming and water gas shift or reversed water gas shift.

The demand for carbon monoxide in the hydroformylation is 1 mol of carbon monoxide/1 mol of ethylene. The amount of ethylene in the product stream of the OCM is n_{OCM}(C₂H₄). Therefore, it is usually necessary to accordingly increase the proportion of carbon monoxide n_{OCM}(CO) already present in the product gas stream after the OCM. The required additional demand n_{Addition}(CO) of carbon monoxide is

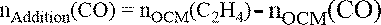

Dry reforming provides the amounts of carbon monoxide n_{DryRef}(CO) und hydrogen n_{DryRef}(H₂). If the additional demand for carbon monoxide is completely covered by the dry reforming, then, in accordance with equation II, the following applies:

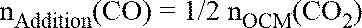

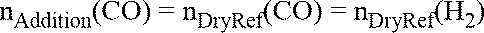

In other words, within the scope of the invention, a gas is considered to be ideal which has the amounts of CO and carbon dioxide defined by equations VI and VIIa in relation to ethylene. This ratio is therefore achieved by inserting equation VIIa in equation VI assuming ideal conversions according to equation II in dry reforming and with a sufficient proportion of hydrogen as in the above exemplary embodiment.

Accordingly, the following applies to the overall composition:

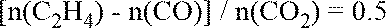

Moreover, if the provision of additional hydrogen n_{Addition}(H₂) is also required by reforming in accordance with equation III for the total reaction I — i.e. when the amounts n_{DryRef}(H₂) and n_{OCM}(H₂) are not sufficient — the following applies here:

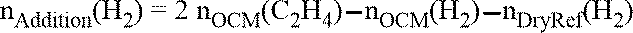

In accordance with the stoichiometry of equation III, carbon monoxide is again formed as a coupling product:

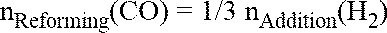

Thus, equation VI is to be extended:

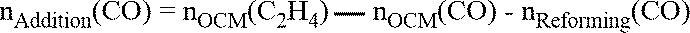

Insertion of equation X in equation XI results in:

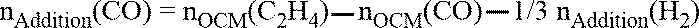

Further inserting of equation IX in equation XII leads to:

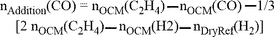

Insertion of equation VII in equation XIII results in equation XIV.

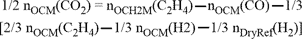

Accordingly, the relative amount of carbon dioxide present in the product gas stream of the oxidative coupling in the idealized case is obtained from equation XIV by rearranging to equation XV:

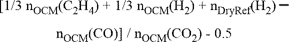

If the ratio according to the equation XV is less than 0.5, there is an excess of carbon dioxide and carbon dioxide must be removed from the process by an appropriate purging stream.

At a ratio greater than 0.5, the amount of carbon dioxide is not sufficient to cover the demand for carbon monoxide, and either an import of carbon dioxide into the dry reforming step in accordance with equation II or an increase in the proportions of hydrogen and carbon monoxide by steam reforming in accordance with equation III and optionally water gas shift in accordance with equation V is required.

As already described, the ratio between hydrogen and carbon monoxide after dry reforming can be adapted as required in particular by a water gas shift.

1. A method for producing a target compound, the method comprising:
providing a starting gas mixture comprising
a paraffin, an olefin, carbon monoxide, and carbon dioxide,
subjecting the olefin in at least a portion of the starting gas mixture is to a hydroformylation process to obtain an aldehyde, wherein the olefin has a carbon chain having a first carbon number and the aldehyde has a second carbon chain having a second carbon number, wherein the second carbon chain is greater by one than the first carbon number, and
removing wherein the carbon dioxide present in the starting mixture at least in part in at least one of the upstream or downstream or combinations of the upstream and downstream relative to the hydroformylation process,
subjecting at least part of the separated carbon dioxide to a dry reforming process with methane to obtain carbon monoxide, and using at least part of the carbon monoxide obtained in the dry reforming process in the hydroformylation process.

2. The method of claim 1, wherein a provision of the starting gas mixture comprises an oxidative coupling of a second paraffin, wherein the second paraffin comprises methane.

3. The method of claim 1, wherein the aldehyde from the hydroformylation process is the target compound, or wherein the aldehyde is further reacted to produce the target compound.

4. The method of claim 3 further comprising:
hydrogenating the aldehyde to produce an alcohol having a third carbon chain having the second carbon number.

5. The method of claim 4, wherein the alcohol is dehydrated to produce a second olefin having a fourth carbon chain having the second carbon number.

6. The method of claim 1, wherein the first carbon number is two and the second carbon number is three.

7. The method of claim 2 further comprising:
subjecting the methane to the oxidative coupling, wherein the methane is at least partially separated from natural gas, and
transferring at least one paraffin is from the natural gas into a post-catalytic steam cracking step downstream of the oxidative coupling.

8. The method of claim 1 further comprising:
obtaining the carbon momoxide abtained in the dry reforming process in a first product mixture, wherein the first product mixture comprises hydrogen.

9. The method of claim 8 further comprising:
subjecting a second product mixture from the dry reforming process to a water gas shift.

10. The method of claim 9, further comprising:
supplying at least one of the first product mixture from the dry reforming process or the second product mixture from the water gas shift or combinations of the first product mixture and the second product mixture to the hydroformylation process, wherein the first product mixture and the second product mixture are at least partially unseparated.

11. The method of claim 1 further comprising:
subjecting at least partially the carbon monoxide and the olefin from the starting gas mixture to the hydroformylation process, wherein the carbon monoxide and olefin are without a preliminary separation from one another.

12. The method of claim 1, comprising:
including a second paraffin in the starting gas mixture,
passing at least part of the second paraffin through the hydroformylation method unreacted,
separating the second paraffin is downstream of the hydroformylation method
using the second paraffin in the provision of the starting gas mixture.

13. The method of claim 1 further comprising:
compressing the starting mixture to a pressure level at which the carbon dioxide is separated off and the hydroformylation process, and
carrying out the dry reforming process at a lower pressure level than the pressure level in the hydroformylation process.

14. The method of claim 1, wherein the method is carried out completely non-cryogenically downstream of the provision of the starting gas mixture and the dry reforming process.

15. A system for producing a target compound, the system configured to:
provide a starting gas mixture which comprises a paraffin, an olefin, carbon monoxide and carbon dioxide, and to subject the olefin in at least a portion of the starting gas mixture to hydroformylation process;
obtain an aldehyde, wherein the olefin has a first carbon chain having a first carbon number and the aldehyde has a second carbon chain having a second carbon number, and wherein the second carbon number is greater by one than the first carbon number,
remove the carbon dioxide present in the starting mixture in a location selected from at least one of upstream or downstream or combinations of upstream and downstream relative to the hydroformylation process, and
subject at least part of the separated carbon dioxide a dry reforming process with methane to obtain carbon monoxide, and to use at least part of the carbon monoxide obtained in the dry reforming process in the hydroformylation process.
